(19) United States
(12) Patent Application Publication
(10) Pub. No.: US 2002/0137751 A1
(43) Pub. Date: Sep. 26, 2002

(54) Method of treatment

(76) Inventor: Timothy John Henkel, King of Prussia, PA (US)

Correspondence Address: GLAXOSMITHKLINE, Corporate Intellectual Property - UW2220, P.O. Box 1539, King of Prussia, PA, 19406-0939, US

(21) Appl. No.: 09/953,736

(22) Filed: Sep. 17, 2001

Related U.S. Application Data
(60) Provisional application No. 60/232,809, filed on Sep. 15, 2000.
(60) Provisional application No. 60/245,744, filed on Nov. 3, 2000.

(51) Int. Cl. A61K 31/496

(52) U.S. Cl. 514/253.08

(57) ABSTRACT

A method of reducing the recurrences of acute exacerbations of chronic bronchitis (AECB) in a patient in need thereof comprising administering a therapeutically effective amount of gemifloxacin, or a pharmaceutically acceptable salt thereof.

### BACKGROUND OF THE INVENTION

The present invention relates to the use of gemifloxacin for reducing the recurrences and/or reducing the severity of recurrences of acute exacerbations of chronic bronchitis (AECB).

Patients with chronic bronchitis frequently experience episodes of exacerbation, characterised by increased cough, increased sputum volume and purulence, and respiratory distress. Annual death rates from chronic bronchitis and its exacerbations in various countries, range from approximately 20 to 80 deaths per 100,000 males aged from 55 to 65 years.

The total direct medical costs of treating AECB have been estimated as at least £396 million in the United Kingdom (UK) (1992/3) and up to $2.3 billion in the USA (1995/6). Patients who are hospitalised account for at least 67% of all costs. AECB is also responsible for a significant loss of working days, 1.54 million, and restricted activity days, 3.63 million, in the USA (1994). Patient well-being and quality of life may also be expected to be affected by AECB. The social, medical and economic consequences of AECB are thus considerable.

The pathogens commonly associated with acute exacerbation of chronic bronchitis (AECB) are Haemophilus influenzae, Streptococcus pneumoniae and Moraxella catarrhalis. In the United States of America (USA), almost 100% of clinical M. catarrhalis isolates produce beta-lactamase, with up to 50% of H. influenzae isolates estimated to produce beta-lactamase by the year 2000. Penicillin-resistant strains of S. pneumoniae have also been identified worldwide. Cross-resistance to other antibacterials such as cephalosporins and macrolides is high among isolates of S. pneumoniae expressing high-level penicillin resistance. Thus, the efficacy of therapy with antibacterials such as the penicillins, cephalosporins, and macrolides may be compromised.

EP 688772 discloses novel naphthyridine carboxylic acid derivatives having antibacterial activity, including anhydrous (R,S)-7-(3-aminomethyl-4-methoxyiminopyrrolidin-1-yl)-1-cyclopropyl-6-fluoro-4-oxo-1,4-dihydro-1,8-naphthyridine-3-carboxylic acid (gemifloxacin).

WO 98/42705 discloses (R,S)-7-(3-aminomethyl-4-syn-methoxyimino- pyrrolidin-1-yl)- 1-cyclopropyl-6-fluoro-4-oxo-1,4-dihydro-1,8-naphthyridine-3-carboxylic acid methanesulfonate (gemifloxacin mesylate) and hydrates thereof including the sesquihydrate.

An acute antimicrobial treatment which rapidly eradicates infecting pathogens in AECB could result in less respiratory distress, lower hospitalisation and re-infection rates and in particular increases the time before the next infection would be desirable as this would provide meaningful clinical benefit and could significantly impact costs as well.

### SUMMARY OF THE INVENTION

The present invention is based on the finding that administration of gemifloxacin results in a reduction of recurrences of AECB requiring antimicrobial therapy compared to conventional methods currently used for the treatment of AECB, e.g. treatment with macrolides such as clarithromycin.

According to the invention there is provided a method of reducing the recurrences of acute exacerbations of chronic bronchitis (AECB) in a patient, e.g. a human, in need thereof comprising administering a therapeutically effective amount of gemifloxacin, or a pharmaceutically acceptable salt thereof.

According to the invention there is provided a method of reducing the severity of recurrences of acute exacerbations of chronic bronchitis (AECB) in a patient, e.g. a human, in need thereof comprising administering a therapeutically effective amount of gemifloxacin, or a pharmaceutically acceptable salt thereof.

The salt of gemifloxacin used in the methods of the invention is preferably gemifloxacin mesylate, in particular gemifloxacin mesylate sesquihydrate.

According to the invention there is also provided the use of gemifloxacin, or a pharmaceutically acceptable salt thereof, in the manufacture of a medicament for reducing the recurrences of acute exacerbations of chronic bronchitis (AECB).

According to the invention there is also provided the use of gemifloxacin, or a pharmaceutically acceptable salt thereof, in the manufacture of a medicament for reducing the severity of recurrences of acute exacerbations of chronic bronchitis (AECB).

### DETAILED DESCRIPTION OF THE INVENTION

In the methods of the invention gemifloxacin, or a pharmaceutically acceptable salt thereof, may be administered to a patient as an acute treatment i.e. whilst the patient is experiencing an AECB. Alternatively, gemifloxacin, or a pharmaceutically acceptable salt thereof, may be administered to a patient as an elective treatment i.e. before the patient experiences an AECB. Elective treatment may be particularly suitable for those patients that are known to be at risk of AECB. A particular group of patients which may be mentioned in this respect are those with chronic obstructive pulmonary disease (COPD) at increased risk of repeat exacerbations. Elective treatment may be performed at any time when a patient is considered at risk of developing an AECB, for example, if the patient is going to be exposed to conditions likely to cause an AECB. Thus, for example, elective treatment may be performed at the beginning of the respiratory season i.e. in the autumn in the US and Europe.

For use in the methods of the invention gemifloxacin, or a pharmaceutically acceptable salt thereof, will normally be formulated into a pharmaceutical composition in accordance with standard pharmaceutical practice.

Gemifloxacin, or a pharmaceutically acceptable salt thereof, may conveniently be administered orally, e.g. as tablets or capsules, or parenterally e.g. intravenously. Gemifloxacin, or a pharmaceutically acceptable salt thereof, may be administered in conventional dosage forms prepared by combining them with standard pharmaceutical carriers according to conventional procedures known to those skilled in the art.

For the methods of use disclosed the daily dosage regimen, e.g. optimal quantity and spacing of individual dosages of gemifloxacin, or a pharmaceutically acceptable salt thereof, will be readily determined by those skilled in the art. For example gemifloxacin may be administered orally at a dose of 320 mg (calculated as the free base) once daily for 5 days.

All publications, including but not limited to patents and patent applications, cited in this specification are herein incorporated by reference as if each individual publication were specifically and individually indicated to be incorporated by reference herein as though fully set forth.

### EXAMPLES

The invention is illustrated by the following examples. However, it should be understood that the examples are intended to illustrate but not in any manner limit the scope of the invention.

### Example 1

### Gemifloxacin long-term outcomes in bronchitis exacerbations (GLOBE) study

### SUMMARY

Background: Few health outcome studies have been reported for antimicrobial treatment of acute infections. In one study, trends favoring ciprofloxacin versus ‘usual care’in terms of clinical outcomes were found (Grossman, et al., Chest, 113:131-141 (1998)). In this double-blind, prospective long-term study, patients who enrolled into a study comparing 320 mg gemifloxacin (GEMI) once daily (o.d.)/5 days with 500 mg clarithromycin (CLARI) twice daily (b.d.)/7 days for the treatment of AECB, and agreed at enrollment to continue in the study for up to 26 weeks, were assessed for clinical status and use of health care resources.

Methods: Clinical assessments were performed at screening (day 0), week 4-5 (end of acute study), weeks 12 and 26 and by telephone at weeks 8, 17 and 21.

Results: 438 patients participated, 214 GEMI (mean age 58.5 years, 50% male) and 224 CLARI (mean age 57.6 years, 55% male). Prior systemic steroid use, smoking history and exacerbation history were similar in the two groups. The proportion of patients whose initial AECB resolved and who had experienced no further recurrences requiring antibiotics by week 26 was 71.0% (120/169) in GEMI-treated patients compared with 58.5% (100/171) for CLARI-treated patients (p =0.016). The number of patients hospitalized for RTI-related events over the study period were 5/214 and 14/224 for the GEMI and CLARI groups, respectively (p=0.059).

Conclusions: GEMI (320 mg o.d./5 days) when given as an acute treatment for AECB was superior to CLARI (500 mg b.d./7 days) in preventing further exacerbations, and reduced the number of patients hospitalized for RTI, during a 6 month period following start of therapy. This is the first study to demonstrate such findings and has significant implications for patient care and health outcomes.

### Introduction

The social, medical, and economic consequences of AECB are considerable (Grossman, et al., Chest, 112(6):3108-3138 (1998); Neideram, et al., Clinical Therapy, 21:576-591 (1999); Pechere, et al., Journal of Antimicrobial Chemotherapy, 45(Supp. B):19-24 (2000)). However, there have been few outcomes studies reported for antimicrobial treatment of AECB. Recent studies have assessed long-term clinical benefits following initial antimicrobial therapy for AECB (Grossman, et al., Chest, 113:131-141 (1998); Chodosh, et al., Clinical Infectious Diseases, 27:730-738 (2000). Although encouraging trends favoring quinolone over macrolide therapy (Chodosh, et al., Clinical Infectious Diseases, 27:730-738 (1998) or usual care (Grossman, et al., Chest, 113:131-141 (1998) were observed, neither provided conclusive evidence of long-term outcome benefits.

GLOBE was a prospective study to evaluate the long-term health economic and clinical outcomes of AECB treatment with either clarithromycin (CLARI) or the enhanced-affinity fluoroquinolone, gemifloxacin (GEMI). GLOBE ran concurrently with an acute treatment safety and efficacy study (Wilson, et al., Abstracts of the 40th Interscience Conference of Clinical Microbiology and Infectious Diseases, Toronto, Canada, Abstract 815 (2000)). In the acute treatment safety and efficacy study (Wilson, et al., Abstracts of the 40th Interscience Conference of Clinical Microbiology and Infectious Diseases, Toronto, Canada, Abstract 815 (2000)). 5 days of GEMI treatment was as effective as 7 days of treatment with CLARI. However, higher bacteriological success rates for GEMI were noted at all study visits and were significantly higher in the GEMI group at the late follow-up visit on days 28-35 following study entry (Wilson, et al., Abstracts of the 40th Interscience Conference of Clinical Microbiology and Infectious Diseases, Toronto, Canada, Abstract 815 (2000)). GEMI treatment also resulted in a significantly faster time to H. influenzae eradication in a subset of patients who underwent daily sputum cultures (p=0.02) (Wilson, et al., Abstracts of the 40th Interscience Conference of Clinical Microbiology and Infectious Diseases, Toronto, Canada, Abstract 815 (2000)).

### Methods

Study Design:

Prospective, 26 week, double-blind, observational parallel/follow-on study to an acute treatment efficacy and safety study (study 068) (Wilson, et al., Abstracts of the 40th Interscience Conference of Clinical Microbiology and Infectious Diseases, Toronto, Canada, Abstract 815 (2000)) in which patients randomly received a single course of either 5 days of oral GEMI 320 mg once daily (o.d.) or 7 days of oral CLARI 500 mg twice daily (b.d.) for AECB (Wilson, et al., Abstracts of the 40th Interscience Conference of Clinical Microbiology and Infectious Diseases, Toronto, Canada, Abstract 815 (2000)).

All patients entering study 068 at centers in the USA and Canada were asked to participate in the GLOBE study.

Patients were assessed at screening (visit 1) at day 28-35 (visit 2) then at weeks 12 (visit 3) and 26 (visit 4) and by telephone at weeks 8, 17 and 21.

The GLOBE study was not prospectively powered to detect clinically and economically relevant differences between treatments as the sample size was dependent on the number of patients in study 068 agreeing to participate.

Patient Assessment:

A total of 438 patients were included in the GLOBE study (214 GEMI, 224 CLARI) 386 in the USA and 52 in Canada.

Information was collected on: recurrent episodes of AECB requiring antimicrobial treatment; use of medical resources related to respiratory tract infection (RTI), including RTI-related:- hospitalizations, physician visits and antimicrobial therapy; time off work and usual activities; and negative impact on performance at work or during usual activities.

Health Related Quality of Life (HQRL) information was also collected using the St George's Respiratory Questionnaire (SGRQ) (Jones, et al., American Review of Respiratory Disease, 145:1321-1327 (1992); Jones, et al., Respiratory Medicine, 85 (Supp. B):25-31 (1991).

Data Analysis:

Clinical outcomes were analyzed for patients in the intention to treat population (ITT) who were available for assessment at 26 weeks (visit 4).

Medical resource utilization was analyzed on cumulative data across all patients in the ITT population.

### Results

Patient Population:

The demographic and clinical characteristics of the two treatment populations were similar (Table 1). All 438 patients were included in the ITT population. Similar proportions of patients were withdrawn from the study: 21.5% (GEMI) and 23.7% (CLARI). The most frequent reason for withdrawal was ‘lost to follow-up’ (12.6%, GEMI and 15.2%, CLARI).

TABLE 1
Demographic and Baseline Clinical Characteristics (ITT Population)

| Characteristic | GEMI (N = 214) | CLARI (N = 224) |
|---|---|---|
| Age, years | | |
| mean (SD) | 58.5 (11.8) | 57.6 (11.8) |
| range | 37-88 | 39-90 |
| Sex, n female (%) | 106 (49.5) | 101 (45.1) |
| Race, n caucasian (%) | 188 (87.9) | 205 (91.5) |
| Duration of CB, years | | |
| n | 213 | 224 |
| mean (SD) | 12.7 (12.1) | 12.4 (11.4) |
| range | 2.0-65.1 | 1.8-66.2 |
| AECBs in last year, n (%)^{a} | | |
| 0 | 41 (19.2) | 40 (17.9) |
| 1 to 4 | 143 (66.8) | 158 (70.5) |
| >4 | 29 (13.6) | 26 (11.6) |
| unknown | 1 (0.5) | 0 |
| Supplemental oxygen, n (%) | 21 (9.8) | 14 (6.3) |
| Systemic steroids in last year, | 54 (25.2) | 55 (24.6) |
| n (%) | | |
| Number of pack years patient has | | |
| smoked, n (%) | | |
| 0 | 34 (15.9) | 40 (17.9) |
| >0-30 | 88 (41.1) | 86 (38.4) |
| >30 | 91 (42.5) | 98 (43.8) |
| unknown | 1 (0.5) | 0 |
| Smoked in last month, n (%) | 95 (44.4) | 107 (47.8) |

^{a}Treated with antimicrobials

AECB Recurrence:

Significantly fewer patients experienced AECB recurrence requiring treatment with an antimicrobial by week 26 in the GEMI group compared with those receiving CLARI (treatment difference=12.5%, 95% CI=2.5%, 22.5%; chi-squared test: p=0.016).

(Table 2).

There were 30% fewer patients with a recurrence of AECB in the GEMI group compared with those receiving CLARI (29.0% vs. 41.5%).

| | | | Difference | |
|---|---|---|---|---|
| | % patients with no recurrences | | GEMI-CLARI, | |
| Visit | GEMI, % (n/N) | CLARI, % (n/N) | % (95% CI) | p value^{a} |
| 2 | 87.1 (176/202) | 80.8 (173/214) | 6.3 (−0.7, 13.3) | 0.081 |
| 3 | 80.9 (148/183) | 74.4 (131/176) | 6.4 (−2.2, 15.1) | 0.143 |
| 4 | 71.0 (120/169) | 58.5 (100/171) | 12.5^{b} (2.5, 22.6) | 0.016 |

^{a}Chi-squared test.
^{b}Statistically significant difference gemifloxacin over clarithromycin

RTI-related Hospitalizations:

The cumulative number of patients hospitalized for RTI-related conditions over the 26 week study period was numerically lower in the GEMI group; 5 (2.3%) compared with 14 (6.3%) for CLARI (treatment difference=-3.91%, 95% CI-7.67%, -0.15%; Fishers exact test: p=0.059).

The cumulative number of RTI-related hospitalizations across all visits was numerically lower in the GEMI group; 7 (3.3%) compared with 16 (7.1%) for CLARI (treatment difference=−3.87%, 95% CI=−8.00%, 0.26%; Fishers exact test: p=0.087).

Whilst no statistical difference was found between the two treatment groups, in terms of median RTI-related hospitalization days, important numerical and potential economic differences were observed: 20 days per 100 patients for GEMI versus 37 days per 100 patients for CLARI (Table 3)

TABLE 3
Cumulative number of RTI-Related Hospitalization Days during the
26-week study period (ITT Population)

| | | | | Difference^{a} | |
|---|---|---|---|---|---|
| Treatment | n | Median (range) | Mean (SD) | (95% CI) | p value^{b} |
| GEMI | 214 | 0 (0-16) | 0.20 (1.43) | 0 (0,0) | 0.526 |
| CLARI | 224 | 0 (0-11) | 0.37 (1.60) | | |

^{a}Median difference for gemifloxacin-clarithromycin.
^{b}Mann-Whitney test

### Conclusions

The GLOBE data confirms that the choice of acute antimicrobial treatment for AECB can impact long-term patient outcomes.

Gemifloxacin produced superior long-term clinical outcomes compared to clarithromycin in AECB.

Significantly more patients on gemifloxacin remained free of recurrence at long-term follow-up compared with patients on clarithromycin.

Fewer patients on gemifloxacin were hospitalized, leading to fewer hospitalizations in total.

These results have important implications for patient management in AECB.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

What is claimed is:

1. A method of reducing the recurrences of acute exacerbations of chronic bronchitis (AECB) in a patient in need thereof comprising administering a therapeutically effective amount of gemifloxacin, or a pharmaceutically acceptable salt thereof.

2. The method according to claim 1 comprising administering a therapeutically effective amount of gemifloxacin mesylate.

3. The method according to claim 2 comprising administering a therapeutically effective amount of gemifloxacin mesylate sesquihydrate.

4. The method according to claim 1 wherein gemifloxacin, or a pharmaceutically acceptable salt thereof, is administered as an acute treatment.

5. The method according to claim 1 wherein gemifloxacin, or a pharmaceutically acceptable salt thereof, is administered as an elective treatment.

6. The method according to claim 1 wherein gemifloxacin is administered orally at a dose of 320 mg (calculated as the free base) once daily for 5 days.

7. The method according to claim 1 wherein the patient is suffering from chronic obstructive pulmonary disease.

8. A method of reducing the severity of recurrences of acute exacerbations of chronic bronchitis (AECB) in a patient in need thereof comprising administering a therapeutically effective amount of gemifloxacin, or a pharmaceutically acceptable salt thereof.

9. The method according to claim 8 comprising administering a therapeutically effective amount of gemifloxacin mesylate.

10. The method according to claim 9 comprising administering a therapeutically effective amount of gemifloxacin mesylate sesquihydrate.

11. The method according to claim 8 wherein gemifloxacin, or a pharmaceutically acceptable salt thereof, is administered as an acute treatment.

12. The method according to claim 8 wherein gemifloxacin, or a pharmaceutically acceptable salt thereof, is administered as an elective treatment.

13. The method according to claim 8 wherein gemifloxacin is administered orally at a dose of 320 mg (calculated as the free base) once daily for 5 days.

14. The method according to claim 8 wherein the patient is suffering from chronic obstructive pulmonary disease.
